(19) United States
(12) Patent Application Publication
(10) Pub. No.: US 2025/0065054 A1
(43) Pub. Date: Feb. 27, 2025

(54) A subassembly of a medicament delivery device

(71) Applicant: SHL Medical AG, Zug (CH)

(72) Inventor: Daniel Carlsson, Nacka Strand (SE)

(21) Appl. No.: 18/688,446

(22) PCT Filed: Aug. 24, 2022
(86) PCT No.: PCT/EP2022/073615

§ 371 Date: Mar. 1, 2024

(30) Foreign Application Priority Data
Sep. 6, 2021 (EP) 21195095.1

(51) Int. Cl. A61M 5/315; A61M 5/20; A61M 5/32

(52) U.S. Cl. CPC A61M 5/3158; A61M 5/2033; A61M 5/3204; A61M 2005/2026

(57) ABSTRACT

The present disclosure provides a subassembly of a medicament delivery device. The subassembly includes a housing, a plunger rod, and a support structure connected to the housing and arranged radially outside the plunger rod. The support structure includes a distal abutment surface portion. The support structure is further configured to deflect between a radial inner position, in which proximal displacement of the plunger rod is restricted, and a radial outer position for allowing proximal displacement of the plunger rod. The subassembly further includes a sleeve portion arranged to distally displace from an initial biased proximal position to a distal position. The sleeve portion and the support structure have interacting contact surfaces extending in the radial direction. The contact surface of the sleeve portion faces in a distal direction, while the contact surface of the support structure faces in a proximal direction.

### CROSS-REFERENCE TO RELATED APPLICATIONS

The present application is a U.S. National Phase Application pursuant to 35 U.S.C. § 371 of International Application No. PCT/EP2022/073615 filed Aug. 24, 2022, which claims priority to European Patent Application No. 21195095.1 filed Sep. 6, 2021. The entire disclosure contents of these applications are herewith incorporated by reference into the present application.

### TECHNICAL FIELD

The present disclosure generally relates to a subassembly of a medicament delivery device for expelling medicament from a medicament container structure.

### BACKGROUND

Medicament is conventionally expelled from a medicament delivery device to a so-called dose delivery site, e.g. through the skin of a person in need of the medicament. The medicament delivery device may generally comprise a medicament container that contains the medicament. Typically, the medicament container is arranged within a housing and further expelled when exposed to an activation force from e.g. a drive assembly, including a plunger rod.

The medicament delivery device can be arranged in several different configurations depending on type of medicament, type of user etc. For example, a needle can be directly arranged on the medicament container, whereby a needle cover is arranged around the needle in a non-use state of the medicament delivery device so as to protect the needle from exposure. The medicament container with the needle and the drive assembly are generally displaceably arranged in a housing.

One area of interest for automatic injectors is the human aspect of handling the injector in a safe and reliable manner. By way of example, automatic injectors should allow the patient to hold and use the injector in an ergonomic way to permit a quick penetration and efficient injection at different locations on the body, such as around the waist and/or on the backside of the waist.

Sometimes the patient may not see the injector at those locations and thus need to be able to hold the injector without having to change grip. By removing the action of pushing a button or making them automatically fired, the patient may be free to hold the device in a more comfortable manner.

However, medicament delivery devices are still in need of further improvements. For example, there is a desire for a more intuitive and ergonomic activation of the medicament delivery device.

### SUMMARY

An object of the present disclosure is thus to provide a subassembly of a medicament delivery device, which at least partially solves the above described needs.

According to a first aspect of the present disclosure, there is provided a subassembly for a medicament delivery device. The subassembly comprises a housing; a plunger rod arranged to be displaced between a distal releasable and biased locking position and a proximal position; a support structure coaxially connected to the housing and arranged radially outside the plunger rod, the support structure having a distal abutment surface portion configured to releasably support a distal part of the plunger rod, the distal abutment surface portion projecting in a radial direction towards a longitudinal centre axis, the support structure further being configured to deflect between a radial inner position, in which proximal displacement of the plunger rod is restricted, and a radial outer position for allowing proximal displacement of the plunger rod; and a sleeve portion arranged to de distally displaced from an initial biased proximal position to a distal position. The sleeve portion comprises a proximal end arranged to cooperate with a distal end of a needle cover of the medicament delivery device, such that a distal displacement of the needle cover effects a distal displacement of the sleeve portion from the initial biased proximal position to the distal position.

The sleeve portion and the support structure further comprise interacting contact surfaces extending in the radial direction, the contact surface of the sleeve portion facing in the distal direction, while the contact surface of the support structure facing in a proximal direction.

Further, upon a movement of the sleeve portion by the needle cover towards the distal position, the contact surface of the sleeve portion exerts a force on the contact surface of the support structure causing the support structure to deflect from the radial inner position to the radial outer position, such that the plunger rod is released from the distal releasable and biased locking position.

By the arrangement of the proposed subassembly, there is provided a simple, yet reliable and efficient way of activating the medicament delivery device. That is, the configuration of the subassembly provides a combination of a needle cover and an internal sleeve portion arranged to be used as an activation system when the needle cover is compressed in a distal direction.

To this end, the arrangement of the proposed subassembly allows for removing a required user step compared to hitherto know prior art medicament delivery devices using an activation button. Further details on the arrangement and the configuration of the components providing additional advantages will be further described in the following disclosure.

The subassembly may further comprise a drive spring coaxially arranged between the plunger rod and the support structure. Typically, the sleeve portion is arranged radially outside the plunger rod. In addition, or alternatively, at least a part of the support structure is arranged radially in-between the plunger rod and the sleeve portion.

The support structure may be coaxially connected to the housing and may extend through a circumferential opening of the sleeve portion. The distal abutment surface portion may be arranged radially in-between the plunger rod and the sleeve portion. In this manner, the relative arrangement of the sleeve portion, support structure and plunger rod is provided in a simple, yet reliable manner. The support structure may be an integral part of the housing. As such, there is provided a device that is easy to assemble and manufacture.

The support structure may be made of a flexible material. The flexible material may be a plastic material. The support structure may comprise at least two radially opposite distal abutment surface portions located radially inside an inner circumferential surface of the sleeve portion. In addition, the at least two radially opposite distal abutment surface portions may be located radially inside an inner circumferential surface of the support structure.

The contact surface of the support structure may be a proximal surface of the distal abutment surface portion. In addition, or alternatively, the contact surface of the support structure may be inclined relative to the longitudinal centre axis.

Optionally, at least a portion of the sleeve portion may circumferentially encircle the support structure.

A distal end portion of the sleeve portion may comprise an opening for radially centring the plunger rod about the longitudinal centre axis.

In addition, or alternatively, the sleeve portion may be in a spring-biased arrangement relative to the housing.

In addition, or alternatively, the plunger rod may be in a spring-biased arrangement relative to the support structure. The distal releasable locking position of the plunger rod may be a spring-biased position.

Generally, the housing may have a proximal end and a distal end. Optionally, the subassembly may further comprise a distal end cap arranged to enclose a distal end opening of the housing. Further, the drive spring may be arranged in-between the distal cap and the distal end portion of the sleeve portion.

The contact surface of the sleeve portion may be arranged on a distal portion of the sleeve portion. In addition, or alternatively, the contact surface of the sleeve portion may be arranged on a radially projecting portion of the sleeve portion. The radially projecting portion may further project radially toward the longitudinal centre axis.

According to a second object, there is provided a medicament delivery device comprising the subassembly according to the first aspect. In addition, the medicament delivery device comprises a medicament container structure having a needle, a needle cover arranged in a spring biased arrangement relative to the housing and configured to be displaced in a distal direction relative to the housing from a needle protecting position, in which a needle is disposed within the needle cover, to a distal position. The plunger rod is arranged to act on the medicament container structure and driven by the drive spring when the plunger rod is released from the distal releasable and biased locking position.

Further effects and features of the second aspect are largely analogous to those described above in relation to the first aspect.

The needle cover may be arranged with a longitudinal distance relative to the sleeve portion when the needle cover is in the needle protecting position and the sleeve portion is in the initial biased proximal position. In this manner, the sequence of releasing the plunger rod may be provided in a smoother manner.

Typically, a distal end of the needle cover may be coaxially arranged relative to a proximal end of the sleeve portion.

The components of the medicament delivery device may be arranged in several different ways inside the housing so as to provide the relative displacements of the components as described herein. Typically, the needle cover may be in a spring-biased arrangement relative to the housing. The needle protecting position of the needle cover may be in an initial spring-biased position.

The medicament container structure may comprise medicament delivery device. The medicament container structure may be a syringe. In addition, or alternatively, the medicament delivery device may be a needle

The drive assembly comprising the plunger rod should be construed as an arrangement which is configured to provide an activating force on the medicament container structure during use of the medicament delivery device. By means of the activating force, the medicament is expelled through a needle and into the dose delivery site. The activating force may either be generated by a manual actuation, or automatically when pushing the medicament delivery device to the dose delivery site.

Typically, although strictly not necessary, the medicament delivery device is a single-dose disposable auto-injector. By way of example, the medicament delivery device may be an auto-injector and the medicament delivery member may be an injection needle.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1A is a perspective view of a medicament delivery device in an assembled configuration according to an example embodiment;

FIG. 1B is another perspective view of the medicament delivery device in FIG. 1A;

FIG. 1C is an exploded view of the medicament delivery device in FIG. 1A;

FIG. 2A is a sectional view of a subassembly of the medicament delivery device in FIG. 1A;

FIGS. 2B and 2C are additional sectional views of the subassembly of the medicament delivery device in FIG. 1A;

FIGS. 3A and 3B are enlarged sectional views of parts of the subassembly of the medicament delivery device in FIG. 1A; and

FIG. 4, is a radial cross-sectional view of some components of the subassembly.

### DETAILED DESCRIPTION

The present disclosure will now be described more fully hereinafter with reference to the accompanying drawings, in which exemplifying embodiments are shown. The disclosure may, however, be embodied in many different forms and should not be construed as limited to the embodiments set forth herein; rather, these embodiments are provided by way of example so that this disclosure will be thorough and complete, and will fully convey the scope of the disclosure to those skilled in the art. Like reference character refer to like elements throughout the description.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

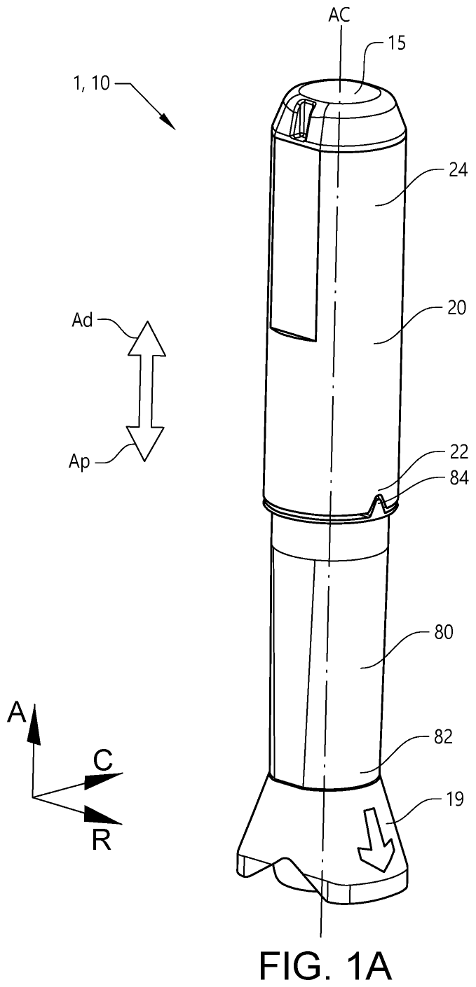

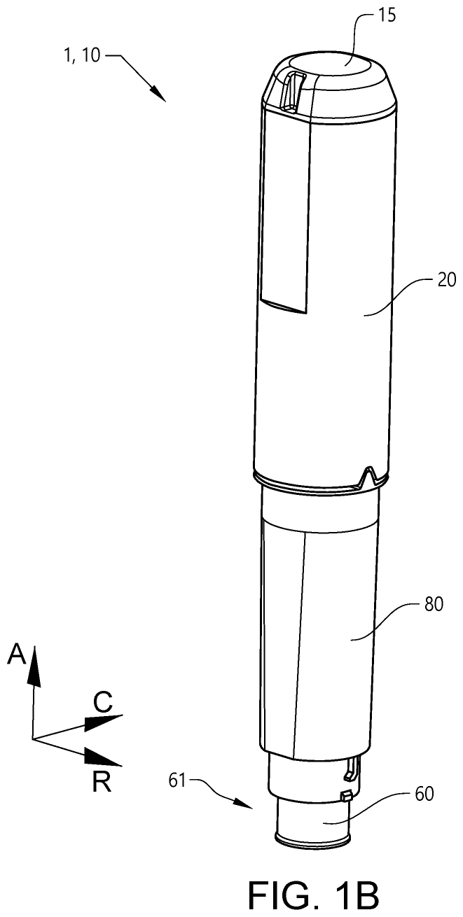

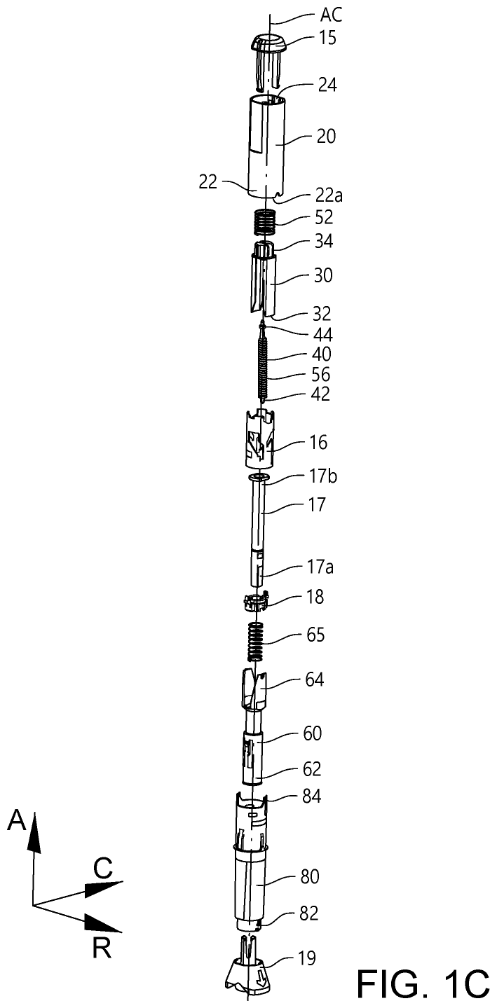

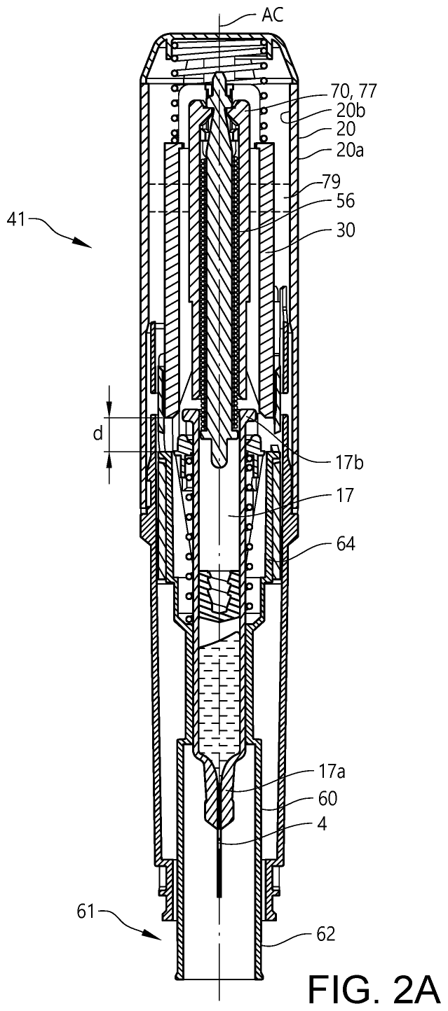

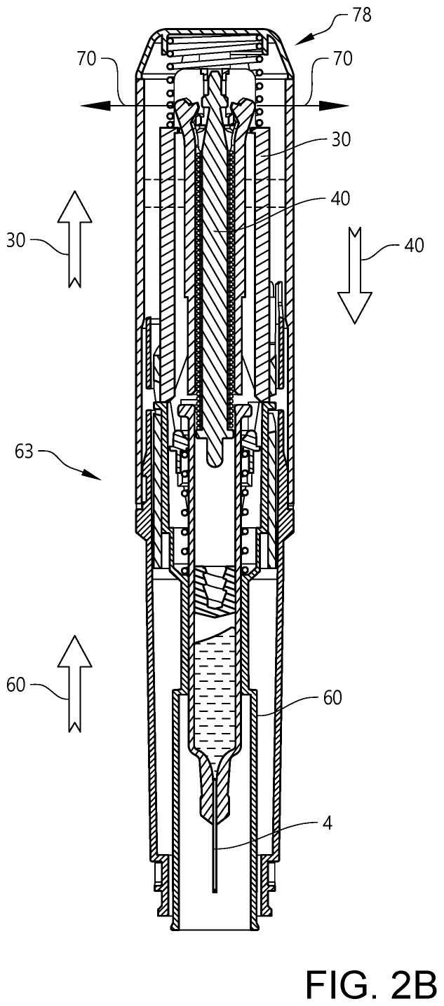

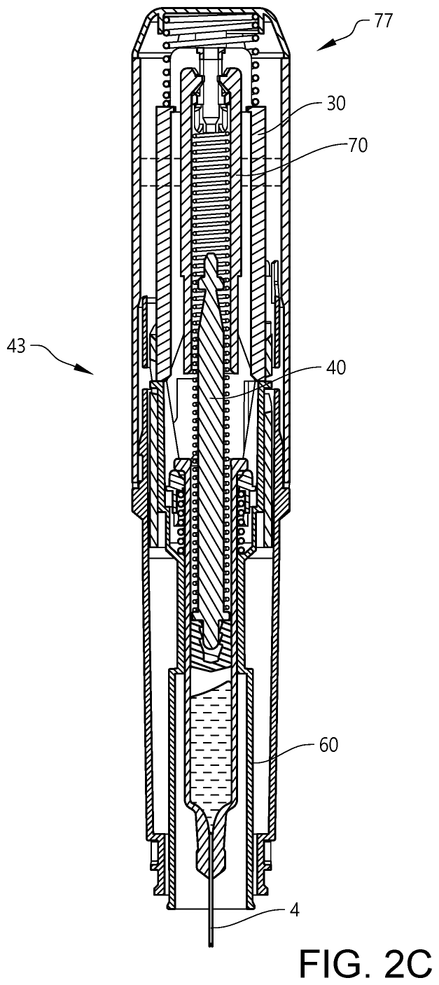

FIGS. 1A to 1C schematically illustrates an example of a medicament delivery device 1 configured to expel medicament from a medicament container structure 17 (shown in FIGS. 2A to 2C). By way of example, the medicament delivery device 1 is a single-dose disposable auto-injector. FIG. 1A illustrates the medicament delivery device 1 in an assembled configuration. As illustrated in FIG. 1A, the medicament delivery device 1 comprises a subassembly 10. The subassembly 10 here comprises a housing assembly having a distal housing 20 and a proximal housing 80. The distal housing 20 and the proximal housing 80 are connected to each other. The distal housing 20 has a proximal end 22 and a distal end 24. The proximal housing 80 has a corresponding proximal end 82 and a corresponding distal end 84. The proximal end 82 of the proximal housing 80 is the end portion of the housing assembly arranged proximal and closest to the dose delivery site during use of the medicament delivery device 1, while the distal end 24 of the housing 20 is the end portion of the housing assembly arranged furthest away from the dose delivery site during use of the medicament delivery device 1.

Accordingly, the distal end 84 of the proximal housing 80 is connected to the proximal end 22 of the distal housing 20. In some examples, the distal housing and the proximal housing may form a common housing. In such examples, the common housing may generally be defined as the housing 20.

In addition, as shown in FIG. 1A, the medicament delivery device 1 comprises a distal end cap 15 and a removable proximal cap 19. The distal end cap 15 is generally securely attached to the distal end 24 of the distal housing 20. The removable proximal cap 19 is generally detachable attached to the proximal end 82 of the proximal housing 80. Each one of the cap 15 and the removable proximal cap 19 may be connected to the respective housing by a snap fit connection, press-fit connection or the like. The cap 15 and the removable proximal cap 19 are commonly known components of the medicament delivery device 1, and thus not further described herein.

Further, it should be noted that the distal housing 20 and the proximal housing 80 can be integral parts and closed by the cap 15. Alternatively, the housing 20 may be a distal housing and the housing 80 may be a proximal housing, as illustrated in FIG. 1A. Alternatively, the housing 20 and the cap 15 may be integral parts and corresponds to a distal housing.

Turning now to FIG. 1B, the medicament delivery device 1 in FIG. 1A is depicted without the removable proximal cap 19. In FIG. 1B, the medicament delivery device 1 is illustrated with a needle cover 60, as further described herein. In FIG. 1C, there is depicted an exploded view of the components of the medicament delivery device 1.

As illustrated in FIGS. 1A to 1C, the medicament delivery device 1 extends in an axial direction A and in a radial direction R. Further, the medicament delivery device 1 has a circumferential extension along a circumferential direction C. The medicament delivery device 1 also has a longitudinal centre axis Ac. In addition, the axial direction A is divided into a proximal direction Ap and a distal direction Ad, as illustrated in FIG. 1A.

As mentioned above, the medicament delivery device 1 comprises the subassembly 10. The subassembly 10 is arranged in the medicament delivery device 1 and comprises the distal housing 20. For ease of reference, the distal housing will in the following be referred to as the housing 20. As illustrated in e.g. FIG. 1C, the housing 20 comprises the proximal end 22 and the distal end 24. In addition, the housing 20 extends a substantial part in the axial direction A and in the radial direction R. The housing 20 is provided in the form of an elongated hollow portion.

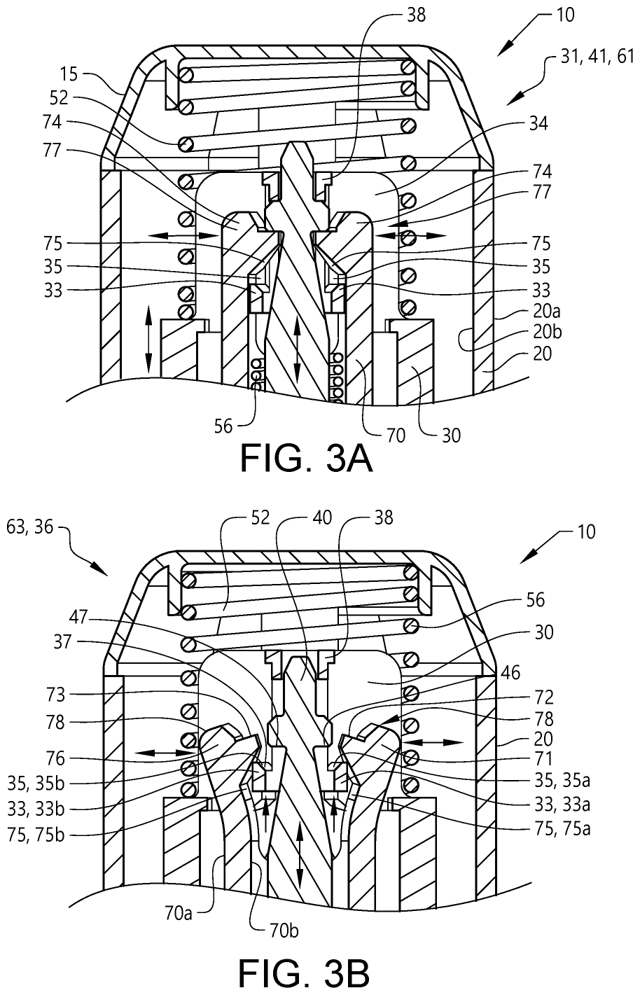

The housing 20 comprises an outer circumferential surface 20a and an inner circumferential surface 20b (see e.g. FIG. 3A). The inner circumferential surface 20b of the housing 20 at least partly defines an inner volume for accommodating the components of the subassembly 10 as described herein.

As may be gleaned from FIG. 1C, the housing 20 comprises an opening 22a at the distal end 22, while the distal end 24 of the housing 20 comprises a corresponding opening (not explicitly indicated in FIG. 1C) for the distal end cap 15.

As illustrated in e.g. FIGS. 1B to 1C and 2A to 2C, the medicament delivery device 1 also comprises a needle cover 60. The needle cover 60 extends a substantial part in the axial direction A and in the radial direction R. The needle cover 60 has a needle cover proximal end 62 and a needle cover distal end 64. The needle cover 60 can be designed in several different manners. By way of example, the needle cover 60 is a hollow cylindrical portion adapted for accommodating a needle 4 and any further moveable parts of a medicament container structure 17.

The needle cover 60 is arranged to displace in the housing 20. In particular, the needle cover 60 is arranged to displace in the distal direction Ad relative to the housing 20 from a needle protecting position 61 (FIG. 2A), in which the needle 4 is disposed within the needle cover 60, to a needle cover distal position 63 (FIG. 2B).

The needle protecting position 61 typically corresponds to a needle cover proximal position, as illustrated in FIGS. 1B, 2A and 3A. The needle cover 60 is here arranged in a spring-biased arrangement relative to the housing 20. The needle protecting position 61 of the needle cover 60 is here also an initial spring-biased position. The initial spring-biased position of the needle cover 60 is provided by a resilient member 65, as shown e.g. in FIG. 1C. By way of example, the resilient member is a spring member 65.

In addition, as illustrated in FIG. 2B, the needle cover distal position 63 is a position where at least a portion of the needle cover 60 is arranged inside or flush with a part of the medicament delivery device 1, e.g. flush with the proximal housing 80. The needle cover distal position 63 further allows the needle 4 to be moved proximally away from the medicament delivery device 1 upon activation of the device, as illustrated in FIG. 2C. In addition, as will be described herein, a displacement of the needle cover 60 to the needle cover distal position 63 allows the needle cover 60 to contact a sleeve portion 30 for operating a plunger rod 40.

Accordingly, as illustrated in e.g. FIG. 1C, the subassembly 10 further comprises a plunger rod 40. Generally, the plunger rod 40 is arranged to act on the medicament container structure 17 for delivery of the medicament. The plunger rod 40 has a plunger rod proximal end 42 and a plunger rod distal end 44.

The plunger rod 40 is arranged to displace inside the housing 20 between a distal releasable and biased locking position 41 (FIGS. 2A and 3A) and a proximal position 43 (FIG. 2C). As illustrated in FIGS. 3A and 3B, the subassembly 10 here comprises a resilient member 56. The resilient member is here a spring member 56. The spring member 56 corresponds to the power spring or drive spring. The spring member 56 is a pre-tensioned drive spring inserted into the plunger rod 40 for displacing the plunger rod 40 in the proximal direction Ap when the plunger rod 40 is released from its distal releasable and biased locking position 41. Accordingly, the distal releasable and biased locking position 41 of the plunger rod 40 is a spring-biased position. When the plunger rod 40 is displaced into the proximal position 43, as shown in FIG. 2C, the plunger rod 40 exerts a force on the medicament container structure 17.

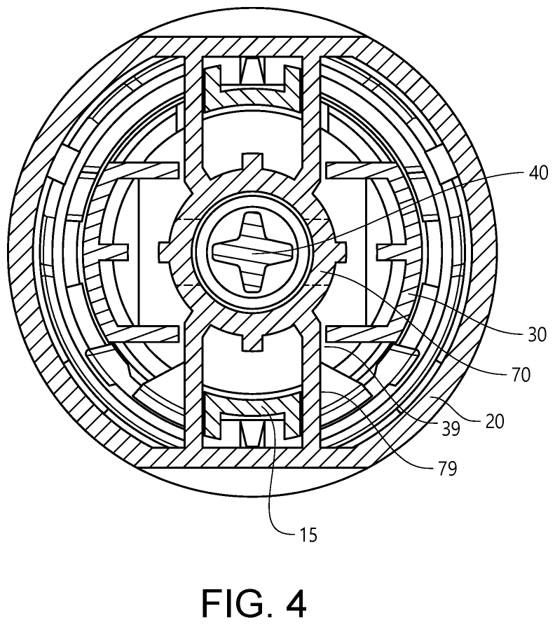

Further, as illustrated in FIGS. 2A to 2C, in combination with FIG. 4, the subassembly 10 comprises a support structure 70 for the plunger rod 40. In this example, the support structure 70 is an integral part of the housing. The support structure 70 is coaxially connected to the housing 20. The support structure 70 projects from an inner side of the housing 20, as illustrated in FIG. 2A and FIG. 4, which is a cross-sectional view of the subassembly 10. The support structure is arranged to the inner circumferential surface 20b via a connection 79. As illustrated in FIG. 4, the connection 79 extends radially from the inner circumferential surface 20b and generally across an opening 39 of a sleeve portion 30, as further described herein.

In other examples, the support structure 70 may be a separate part of the subassembly 10 that is arranged to the inner circumferential surface 20b of the housing 20. Hence, the connection 79 may be an integral part of the housing or an integral part of the support structure 70.

The support structure 70 is made of a flexible material. By way of example, the flexible material is a plastic material having a sufficient flexible characteristic for the purpose of the support structure as described herein.

Turning again to FIGS. 2A to 2c in combination with the enlarged views in FIGS. 3A and 3B, the support structure 70 is arranged radially outside the plunger rod 40. The support structure 70 has an inner circumferential surface 70b and an outer circumferential surface 70a (FIG. 3B). As illustrated in FIG. 3B, the support structure 70 comprises first and second radially opposite distal abutment surface portions 72, 73 that are located radially inside the inner circumferential surface 70b of the support structure 70. In other words, each one of the abutment surface portions 72, 73 is disposed on a distal portion 74 of the support structure 70. Moreover, each one of the abutment surface portions 72, 73 is arranged on radially opposite inner portions of the support structure 70, as illustrated in FIGS. 3A and 3B. As such, the abutment surface portions 72, 73 are arranged radially outside the plunger rod 40.

In addition, each one of the distal abutment surface portions 72, 73 projects in the radial direction R towards the longitudinal centre axis Ac. In other words, the first distal abutment surface portion 72 is arranged at a first radial position and projects in the radial direction R towards the longitudinal centre axis Ac, while the second distal abutment surface portion 73 is arranged at a second opposite radial position and projects in the radial direction R towards the longitudinal centre axis Ac. Each one of the distal abutment surface portions 72, 73 is thus adapted to support the plunger rod 40, as illustrated in FIG. 3A.

By way of example, each one of the distal abutment surface portions 72, 73 are arranged to abut a corresponding radially extending contact surface 46, 47 of the plunger rod 40. The radially extending contact surfaces 46, 47 of the plunger rod 40 are disposed at the distal end 44 of the plunger rod, as depicted in FIGS. 3a and 3B. Each one of the radially extending contact surfaces 46, 47 of the plunger rod 40 projects radially away from the longitudinal centre axis Ac.

Further, the support structure 70 is configured to deflect between a radial inner position 77 (FIG. 3A), in which proximal displacement of the plunger rod 40 is restricted, and a radial outer position 78 (FIG. 3B) for allowing proximal displacement of the plunger rod 40.

The proximal displacement of the plunger rod 40 is restricted by means of the axially opposite arrangement of the corresponding radially extending contact surface 46, 47 of the plunger rod 40 and the distal abutment surface portions 72, 73 of the support structure, as also illustrated in FIG. 3B. As such, the distal abutment surface portions 72, 73 are configured to releasably support a distal part of the plunger rod.

In addition, the plunger rod 40 is here arranged in a spring-biased arrangement relative to a support structure 70. The plunger rod 40 is arranged in the spring-biased arrangement relative to a support structure 70 by means of the arrangement of the distal abutment surface portions 72, 73, the corresponding radially extending contact surface 46, 47 and the resilient member 56, as described above. When the distal abutment surface portions 72, 73 are radially displaced in the radial outer position 78, as described hereinafter, the plunger rod 40 is released from the distal releasable and biased locking position 41 and is thus allowed to displace in the proximal direction Ap towards the medicament container structure 17. To this end, the resilient member 56 is arranged to initiate and drive the movement of the plunger rod 40 in the proximal direction Ap, as generally is known in the art.

Turning again to FIGS. 3A and 3B, the distal abutment surface portions 72, 73 are arranged on two axially projecting elongated members 75, 76 of the support structure 70. Each one of the two axially projecting elongated members 75, 76 projects from the support structure 70 and are generally integral parts thereof. As such, the two axially projecting elongated members 75, 76 correspond to a pair of flexible arms of the support structure 70. As mentioned above, and illustrated in FIGS. 3A and 3B, the flexible arms are capable of flexing radially outwardly relative to the longitudinal centre axis Ac.

It should be readily appreciated that the distal abutment surface portions 72, 73 may be arranged in other ways on the support structure 70 and in different configurations. By way of example, it is sufficient that the support structure 70 comprises one single distal abutment surface portion 72 for the plunger rod 40, which may be arranged as the first distal abutment surface portion 72, as described herein. Alternatively, the single distal abutment surface portion may extend a substantial length in the circumferential direction C so as to support the plunger rod 40.

Turning again to the plunger rod 40 and the medicament container structure 17. As may gleaned from fig. FIGS. 2A to 2C, the plunger rod 40 is arranged to move forward in the proximal direction Ap so as to act on the medicament container structure 17 when the plunger rod 40 is in its proximal position 43 (FIG. 2C). Similar to the other components of the subassembly 10, the medicament container structure 17 is here arranged in a spring-biased configuration. By way of example, the medicament container structure 17 is spring-biased relative to a medicament container support collar 18, as illustrated in FIG. 1C. The medicament container support collar 18 is here an integral part of the subassembly 10. Hence, the medicament delivery device 1 comprises the medicament container support collar 18. As shown in FIG. 2B, the medicament container support collar 18 is arranged radially in-between the medicament container structure 17 and the needle cover 60. Accordingly, the medicament container structure 17 is arranged radially inside the needle cover 60. That is, the medicament container structure 17 is displaceable radially inside the needle cover 60.

The medicament container structure 17 is e.g. a syringe. As illustrated in FIGS. 2A to 2C, a proximal end 17a of the medicament container structure 17 is equipped with a medicament delivery member 4. By way of example, the medicament delivery member is the injection needle 4. The needle 4 is usually protected and surrounded by the needle cover 60. The medicament container structure 17 also comprises a distal end 17b for receiving the plunger rod 40, as illustrated in FIG. 2A. Hence, the distal end 17b has an opening.

Further, the needle cover 60 here comprises a proximal end central opening 60a having a dimension for allowing an arrangement of the removable protective cap 19 to be arranged through the opening to protect the needle 4 prior to use. The cap 19 is generally initially removed from the needle cover 60, as illustrated by FIG. 1A, while the needle cover 60 is allowed to activate the use of the subassembly 10 according to the following description. As may be gleaned from e.g. FIG. 1C, the needle cover 60 also comprises a distal end opening 60b located at the distal end 64.

The needle cover 60 is here spring-biased arranged to the housing 20. By way of example, the needle cover 60 is spring-biased by means of the resilient member 65 biasing the needle cover 60 in an extended position from the proximal end 82 of the proximal housing 80. The extended position from the proximal end of the housing corresponds to the proximal position of the needle cover 60.

As illustrated in FIGS. 1C, 2A to 2C and 3A to 3B, the subassembly 10 also comprises a sleeve portion 30 arranged to distally displace from an initial biased proximal position 31 to a distal position 36. The sleeve portion 30 comprises a proximal end 32 and a distal end 34. The proximal end 32 of the sleeve portion 30 is configured to cooperate with the distal end 64 of the needle cover 60. More specifically, as illustrated by FIGS. 2A to 2C, in conjunction with FIGS. 3A and 3B, the proximal end 32 is arranged to cooperate with the distal end 64 of the needle cover 60, such that a distal displacement of the needle cover 60 from the needle protecting position 61 to the distal position 63 effects a distal placement of the sleeve portion 30 from the initial biased proximal position 31 to the distal position 36.

By the arrangement and configuration of the needle cover 60 and the sleeve portion 30, as illustrated by the FIGS. 2A to 2C, it becomes possible to provide a distal movement of the sleeve portion 30 by a distal movement of the needle cover 60.

The sleeve portion 30 is here in a spring-biased arrangement relative to the housing 20. By way of example, the subassembly 10 comprises a drive spring 52 (such as a resilient member) arranged in-between the distal cap 15 and the distal end portion 34 of the sleeve portion 30, as e.g. illustrated in FIGS. 2A to 2C, and also in FIGS. 3A and 3B.

Moreover, at least a portion of the sleeve portion 30 circumferentially encircles the support structure 70. Also, as illustrated in FIGS. 3A and 3B, the distal end portion 34 of the sleeve portion comprises an opening 38 for radially centring the plunger rod 40 about the longitudinal centre axis Ac.

Typically, the distal end 64 of the needle cover 60 is coaxially arranged relative to the proximal end 32 of the sleeve portion 30. By way of example, as illustrated in FIG. 2A, the needle cover 60 is further arranged with a longitudinal distance d relative to the sleeve portion 30 when the needle cover 60 is in the needle protecting position 61 and the sleeve portion 30 is in the initial biased proximal position 31.

Turning again to FIGS. 3A and 3B, the sleeve portion 30 comprises a pair of contact surfaces 35, 35a, 35b. In addition, the support structure 70 comprising mating contact surfaces 75, 75a, 75b. As such, the sleeve portion 30 and the support structure 70 comprise interacting contact surfaces 35, 75 extending in the radial direction R, respectively.

Each one of the contact surfaces 35, 35a, 35b of the sleeve portion 30 is arranged to face towards the distal direction, while each one of the contact surfaces 75, 75a, 75b of the support structure 70 is arranged to face towards the proximal direction, as illustrated in FIGS. 3A and 3B. Further, as illustrated in FIGS. 3A and 3B, a corresponding contact surface 35a of the sleeve portion 30 faces a corresponding contact surface 75a of the support structure 70. To this end, a distal movement of the sleeve portion 30, causing a distal movement of the contact surfaces 35a, 35b of the sleeve portion, thus exerting a force in the distal direction on the corresponding contact surfaces 75a, 75b of the support structure 70.

The mutual movements of the sleeve portion 30 and the support structure 70 are illustrated by the arrows in FIG. 3B. Accordingly, when the contact surfaces 35a, 35b of the sleeve portion 30 distally displace to exert the force on the contact surface 75a, 75b of the support structure 70, the support structure 70 will flex from the radial inner position 77 (FIG. 3A) to the radial outer position (FIG. 3B). The above movement of the support structure 70 is here provided by the flexible characteristics of the support structure 70 and the fixed attachment 79 of the support structure 70 to the housing 20, as e.g. provided by the arrangement of the support structure 70 to the housing 20 as illustrated in FIG. 2A and FIG. 4.

FIG. 4 is a radial cross-sectional view of how the support structure can be arranged in the housing 20. As illustrated in FIG. 4, the support structure 70 extends radially from the inner circumferential surface 20b of the housing and through the circumferential opening 39 defined between portions of the sleeve portion 30. Further, the distal abutment surface portions 72, 73 are arranged radially in-between the plunger rod and the sleeve portion.

By way of example, the contact surfaces 35, 35a, 35b of the sleeve portion 30 are arranged on radially projecting portions 33, 33a, 33b of the sleeve portion 30. Each one of the radially projecting portions 33, 33a, 33b of the sleeve portion 30 is arranged on the distal portion 34 of the sleeve portion 30. In addition, each one of the radially projecting portions 33, 33a, 33b of the sleeve portion 30 projects radially toward the longitudinal centre axis Ac.

As illustrated in FIGS. 2A to 2C, and also in FIGS. 3A and 3b, each one of the radially projecting portions 33, 33a, 33b of the sleeve portion 30 projects radially inside the inner circumferential surface 70b of the support structure 70. Further, in this example, each one of the radially projecting portions 33, 33a, 33b is arranged on a radially inner side 37 of the sleeve portion 30, as may be gleaned from FIG. 2A or FIG. 3B.

The displaceable movement of the needle cover 60 and the sleeve portion 30 to effect a radial flex of the support structure 70 is illustrated by the sequences in FIGS. 2A to 2C, and further also by the FIGS. 3A and 3B that further indicate the release of the plunger rod 40 due to the above interaction between the needle cover 60, the sleeve portion 30 and the support structure 70. In other words, as illustrated by the arrows in FIG. 2B, a movement of the needle cover 60 from the proximal position 61 to the distal position 63 pushes the sleeve portion 30 from its proximal position 31 to its distal position 36, whereby the support structure 70 flexes from the radial inner position 77 to the radial outer position 78. To this end, the plunger rod 40 can be released from the distal releasable and biased locking position 41 and allowed to move in the proximal direction Ap to its proximal position 43, as illustrated in FIG. 2C.

In other words, upon a movement of the sleeve portion 30 by the needle cover 60 to the distal position 63, the contact surfaces 35, 35a, 35b of the sleeve portion 30 exert a force on the corresponding contact surfaces 75, 75a, 75b of the support structure 70 causing the support structure 70 to deflect from the radial inner position 77 to the radial outer position 78, such that the plunger rod 40 is released from the distal releasable and biased locking position 41.

While the surfaces of the support structure may be arranged in several different manners, the contact surfaces 75, 75a, 75b of the support structure 70 are here proximal surfaces of the corresponding distal abutment surface portions 72, 73.

By way of example, the contact surface 75, 75a, 75b of the support structure 70 are inclined relative to the longitudinal centre axis Ac. In addition, or alternatively, the contact surface 35, 35a, 35b of the sleeve portion 30 are perpendicularly oriented relative to the longitudinal centre axis Ac.

Optionally, the subassembly 10 comprises a rotator 16, as illustrated in FIG. 1C. The rotator 16 is generally a rotational member connected to the needle cover 60. The rotator is further configured to rotate and interact with the needle cover 60 as the syringe collar 18 is moved in the proximal direction, when it has been pressed against an injection site. The rotator 16 is a common element of a medicament delivery device 1 and thus not further described herein.

According to some variations, the drive member of the plunger rod and the medicament container could for example be motor driven or manually driven instead of having an automatic spring-loaded structure.

The disclosure has mainly been described above with reference to a few examples. However, as is readily appreciated by a person skilled in the art, other embodiments than the ones disclosed above are equally possible within the scope of the disclosure, as defined by the appended claims.

1-15. (canceled)

16. A subassembly for a medicament delivery device, the subassembly comprising:
a housing;
a plunger rod arranged to be displaced between a distal releasable and biased locking position and a proximal position;
a support structure coaxially connected to the housing and arranged radially outside the plunger rod, the support structure having a distal abutment surface portion configured to releasably support a distal part of the plunger rod, the distal abutment surface portion projecting in a radial direction towards a longitudinal center axis, the support structure further being configured to deflect between a radial inner position, in which proximal displacement of the plunger rod is restricted, and a radial outer position for allowing proximal displacement of the plunger rod; and
a sleeve portion arranged to be distally displaced from an initial biased proximal position to a distal position, the sleeve portion having a proximal end arranged to cooperate with a distal end of a needle cover of the medicament delivery device, such that a distal displacement of the needle cover effects a distal displacement of the sleeve portion from the initial biased proximal position to the distal position,
wherein the sleeve portion and the support structure have interacting contact surfaces extending in the radial direction, the contact surface of the sleeve portion facing in the distal direction, while the contact surface of the support structure facing in a proximal direction, and
whereby, upon a movement of the sleeve portion by the needle cover towards the distal position, the contact surface of the sleeve portion exerts a force on the contact surface of the support structure causing the support structure to deflect from the radial inner position to the radial outer position, such that the plunger rod is released from the distal releasable and biased locking position.

17. The subassembly according to claim 16, further comprising a drive spring coaxially arranged between the plunger rod and the support structure.

18. The subassembly according to claim 16, wherein the support structure is coaxially connected to the housing and extends through a circumferential opening of the sleeve portion, wherein the distal abutment surface portion is arranged radially in-between the plunger rod and the sleeve portion.

19. The subassembly according to claim 16, wherein the support structure is made of a flexible material.

20. The subassembly according to claim 16, wherein the support structure comprises at least two radially opposite distal abutment surface portions located radially inside an inner circumferential surface of the sleeve portion.

21. The subassembly according to claim 16, wherein the contact surface of the support structure is a proximal surface of the distal abutment surface portion.

22. The subassembly according to claim 16, wherein the contact surface of the support structure is inclined relative to the longitudinal center axis.

23. The subassembly according to claim 16, wherein at least a portion of the sleeve portion circumferentially encircles the support structure.

24. The subassembly according to claim 16, wherein a distal end portion of the sleeve portion comprises an opening for radially centering the plunger rod about the longitudinal center axis.

25. The subassembly according to claim 16, wherein the sleeve portion is in a spring-biased arrangement relative to the housing. 26 (New) The subassembly according to claim 16, wherein the plunger rod is in a spring-biased arrangement relative to the support structure, the distal releasable locking position of the plunger rod being a spring-biased position.

27. The subassembly according to claim 16, further comprising a distal end cap arranged to enclose a distal end opening of the housing.

28. The subassembly according to claim 16, wherein the contact surface of the sleeve portion is arranged on a distal portion of the sleeve portion.

29. The subassembly according to claim 16, wherein the contact surface of the sleeve portion is arranged on a radially projecting portion of the sleeve portion, and wherein the radially projecting portion projects radially toward the longitudinal center axis.

30. A medicament delivery device comprising:
the subassembly according to claim 16;
a medicament container structure having a needle, a needle cover arranged in a spring biased arrangement relative to the housing and configured to be displaced in a distal direction relative to the housing from a needle protecting position, in which a needle is disposed within the needle cover, to a distal position, wherein the plunger rod is arranged to act on the medicament container structure and driven by the drive spring when the plunger rod is released from the distal releasable and biased locking position.
